(19) United States
(12) Patent Application Publication
(10) Pub. No.: US 2017/0212498 A1
(43) Pub. Date: Jul. 27, 2017

(54) MULTI-FUNCTION CONTROL DEVICE AND METHOD OF OPERATION

(71) Applicant: orangedental GmbH & Co. KG, Biberach (DE)

(72) Inventors: Ludwig LAXHUBER, Herrsching (DE); Bernhard PIERNITZKI, Demen (DE)

(21) Appl. No.: 15/481,931

(22) Filed: Apr. 7, 2017

Related U.S. Application Data
(63) Continuation-in-part of application No. 13/812,311, filed on May 29, 2013, now abandoned.

(30) Foreign Application Priority Data
Jul. 29, 2010 (EP) 10007954.0

(51) Int. Cl. G05B 19/409; A61C 1/00; A61B 17/00

(52) U.S. Cl. CPC G05B 19/409; A61B 17/00; A61C 1/0023; G05B 2219/23056; G05B 2219/45167; A61B 2017/00393; A61B 2017/00973; A61B 2560/0223

(57) ABSTRACT

Methods of operating a control device for controlling at least two different functions of an instrument in communication with the control device are provided, comprising for example the following steps: calibrating the control device in a calibration mode by assigning the at least two different functions to at least two different ways of actuating an actuation element; and controlling the instrument in communication with the control device in an actuation mode to perform at least one of the at least two different functions defined in the calibration mode by an actuation of the actuation element. Corresponding devices are also provided.

### CROSS-REFERENCE TO RELATED APPLICATIONS

This application is a continuation-in-part of U.S. patent application Ser. No. 13/812,311, filed on May 29, 2013, which is a U.S. National Stage of PCT/EP2011/003502, filed on Jul. 13, 2011, which claims priority of European Patent Application No. 10007954.0, filed on Jul. 29, 2010, each of which is incorporated herein by reference.

### TECHNICAL FIELD OF THE INVENTION

The present invention relates to a control device and in particular to a foot-actuated control device for the operation and control of medical or dental equipment or instruments. The present invention also relates to a method of operating such a control device.

### BACKGROUND OF THE INVENTION

Dental or medical professionals and practitioners use many instruments that are controlled by foot control systems. For example, surgical cutting instruments, endoscopic tools, irrigation and aspiration tools, dental drills and other handpieces, ultrasonic dental sealers, and dental prophylaxis units can be activated by means of foot control systems. A foot-actuated control system typically includes a device or switch that is placed on the floor within easy reach of the practitioner. The foot switch is used to activate a dental/medical apparatus, which can include an operating base unit in communication with the foot switch. The foot switch is typically connected to the base unit by a connector cable in a “hardwired” system. Alternatively, remote, “wireless” foot-actuated control systems, which do not use a connector cable, can be used to activate the base unit in some instances. A flexible, instrument cable connects the dental/medical instrument, for example, a dental handpiece, to the base unit. The dental or medical practitioner activates the base unit and the dental/medical instrument connected thereto by depressing the foot switch with his or her foot.

Some conventional foot switches are referred to as multi-position or multi-operation switches, i.e., switches that can control or trigger more than one function of an instrument in communication therewith. An operator depresses the pedal of the foot switch to a certain position, and this action causes the dental/medical instrument to operate in a specific mode. The particular operational mode is based on the position of the foot switch pedal. For example, with a two-position foot switch, a dental practitioner can depress the pedal to a first position so that water flows through the handpiece for rinsing the teeth of a patient. Then, the pedal of the foot switch can be depressed to a second position so that a cleaning spray flows through the handpiece for cleaning the teeth.

Foot-actuated control systems provide several advantages. First, the foot switch device is easy to use and efficient. The dental/medical professional or practitioner can activate the instrument in communication with the foot switch and optionally a base unit by simply depressing the foot switch with his or her foot. Secondly, the dental/medical practitioner's hands are kept free when working with a foot switch device. The practitioner thus can handle other instruments and accessories while treating the patient. Thus, the practitioner is better able to concentrate on performing the required dental/medical procedure.

Foot switch devices can have a wide variety of structures. For example WO 2007/084605 discloses a foot switch device for activating a dental or medical treatment instrument. The foot switch device includes a base plate, a central housing attached to the base plate, and an upper, moveable cover mounted on the housing. A connecting collar is attached to the upper cover for retaining the cover on the housing while allowing the cover to move upwardly and downwardly relative to the housing. An actuating plunger is sildably mounted in the central housing below the cover. The central housing also contains a first electrical switch for transmitting a first signal to the instrument, and a second electrical switch for transmitting a second signal to the instrument. An operator depresses the upper cover with his or her foot to move the slidable plunger which, in turn, activates the switching mechanisms and controls the operation of the dental or medical instrument.

EP 1462906 discloses a foot switch device or regulator, especially for dental equipment. The foot regulator comprises a base part relative to which a regulating part of the foot regulator may be rotated and/or displaced in a radial direction, as well as means for detecting rotation and/or displacement. Moreover, the foot regulator comprises means for detecting the instantaneous position of the regulator, means for detecting relative movement relative to this position as well as detectable reference means for determining position and/or movement, wherein the detectable reference means for determining position and/or movement are formed by a pattern, diagram or elevations having a form of lattice structure. EP 1462906, moreover, discloses a method of controlling dental equipment, wherein the control is performed on the basis of a reading or detection of the instantaneous position of the foot regulator relative to a given zero point. The control takes place by rotation and/or radial displacement of one or more regulating rings.

The above described as well as other conventional foot switch devices that provide for more than one operation mode, i.e., can activate more than one function of an instrument in communication therewith, have the following drawback. Often, a dental/medical professional or practitioner, who is either standing on the ground or sitting on a chair, will have to change his location with respect to a patient, for instance, in order to obtain a different view of a region being examined or to examine different body parts of the patient. In doing so the dental/medical professional in a lot of cases will also change his position with respect to a foot switch located at a certain position on the floor. In order to still be able to operate any dental/medical instrument or equipment controlled by the foot switch the dental/medical professional will have to adjust the position of the foot switch to be accessible from his current position. Often the dental/medical professional achieves this by “dragging” the foot switch device along the floor by using his foot. In doing so the relative angular position of the foot switch with respect to the dental/medical professional will often change so that in most cases the dental/medical professional will also have to manually adjust the relative angular position of the foot switch in order to be able to access the whole functionality provided by the foot switch. Having to adjust the relative angular position of the foot switch each time the dental/medical professional changes his position with respect to the foot switch is cumbersome and distracts the dental/medical professional's attention from the patient.

An object of the present invention is to provide an improved control device and in particular an improved foot switch that does not have the above outlined drawbacks. A further object of the present invention is to provide for a method of operating such an improved control device.

### SUMMARY OF THE INVENTION

The above objects are achieved according to a first aspect of the invention by a method of operating a control device for controlling at least two different functions of an instrument in communication with the control device. The method comprises the following steps: calibrating the control device in a calibration mode by assigning the at least two different functions to at least two different ways of actuating an actuation element; and controlling the instrument in communication with the control device in an actuation or control mode to perform at least one of the at least two different functions defined in the calibration mode by an actuation of the actuation element.

In an embodiment of the invention, the at least two different functions are assigned to the at least two different ways of actuating the actuation element by an actuation of the actuation element.

In an embodiment of the invention, the at least two different functions are assigned to the at least two different ways of actuating the actuation element by positioning a user's foot relative to the control device.

In an embodiment of the invention, the method further comprises detecting an actuation of the actuation element, wherein the actuation preferably results in a motion of the actuation element towards a base element of the control device floatingly supporting the actuation element.

In an embodiment of the invention, the detection of an actuation is performed by one or more or a plurality of actuation sensors.

In an embodiment of the invention, the method further comprises exerting a force onto the actuation element for defining a reference position of the control device as a basis for assigning the at least two different functions to the at least two different ways of actuating the actuation element and preferably as a basis for one or more subsequent actuations of the actuation element during the actuation mode.

In an embodiment of the invention, the method further comprises exerting a force onto the actuation element at two different locations or at least at two different locations of an actuation surface of the actuation element for actuating the actuation element in the actuation mode.

The one or more or the plurality of actuation sensors preferably sense any actuation of the actuation element and thus, the calibration or the actuation mode can be triggered due to (a) corresponding sensor signal(s).

Preferably one or more of the actuation sensors of the plurality of actuation sensors detect a specific kind of actuation, since the sensors are preferably responsive to the movement of the actuation element, and trigger(s) the actuation or calibration mode.

In the calibration mode, one or more of the plurality of actuation sensors preferably detect the kind of activation or actuation, preferably the kind of movement of the actuation element and allow for calibrating the control device, thereby considering the movement of the actuation element. Then, an assignment between functions and ways of actuating the actuation element is performed, that is, that the at least two different functions are assigned to the at least two different ways of actuating the actuation element.

In the actuation mode, one or more of the plurality of actuation sensors preferably detect the kind of activation or actuation, preferably via a corresponding actuation area or via the different locations as indicated above, of the actuation element, and thus, the instrument in communication with the control device is controlled and the corresponding function is performed.

In an embodiment of the invention, the method further comprises detecting any motion of the control device with respect to a supporting surface.

In an embodiment of the invention, the method further comprises visually indicating to a user whether the control device is operating in the calibration mode or the actuation mode and/or visually indicating to a user the different functions assigned to the actuation element.

In a further embodiment, the visually indication is performed by a plurality of LEDs preferably corresponding in number and position to the plurality of actuation sensors.

The above objects are achieved according to a second aspect of the invention by a method of operating a control device for controlling at least two different functions of an instrument or of at least one instrument in communication with the control device, the control device being operable in a calibration mode and in an actuation mode, the method comprising the following steps:

(a) providing an actuation system for actuating or operating the control device,

(b) providing a sensor system for detecting an actuation of the actuation system,

(c) selecting the calibration mode for defining a relative position between the control device and a user, and,
depending on the relative position between the control device and a user, assigning the at least two different functions to different actuation areas, preferably to at least two different actuation areas of the actuation system, and

(d) selecting the actuation mode for controlling the at least two different functions by the at least two actuation areas.

In an embodiment of the invention, the method further comprises: depending on the relative position between the control device and the user, defining the at least two actuation areas of the actuation system.

In another embodiment, the method further comprises assigning the at least two different functions to the at least two different actuation areas, provided that not more than one function is assigned to one actuation area.

In an embodiment of the invention, the method further comprises actuating the actuation system to select at least one of the following: the calibration mode and the actuation mode.

In a further embodiment, the actuation of the actuation system results in a motion of the actuation system towards a base element of the control device preferably floatingly supporting the actuation system.

In an embodiment of the invention, the method further comprises defining the relative position between the control device and the user by an actuation of the actuation system.

In an embodiment of the invention, the method further comprises defining the relative position between the control device and the user by positioning a user's foot relative to the control device.

In an embodiment of the invention, the method further comprises defining the relative position between the control device and the user by exerting a force onto the actuation system for defining a reference position of the control device as a basis for assigning the at least two different functions to the at least two different actuation areas and preferably as a basis for one or more subsequent actuations of the actuation element during the actuation mode.

In an embodiment of the invention, the method further comprises selecting a predefined assignment pattern from a plurality of predefined assignment patterns in order to define or for defining the at least two actuation areas.

In an embodiment of the invention, the method further comprises exerting a force onto the actuation system preferably at at least one of the at least two actuation areas for actuating the actuation system for controlling the at least two different functions.

In an embodiment of the invention, the detection of an actuation of the actuation system is performed by the sensor system, in particular by one or more or a plurality of actuation sensors.

The one or more or the plurality of actuation sensors preferably sense any actuation of the actuation system, for example of an actuation element, and thus, the calibration or the actuation mode can be triggered due to (a) corresponding sensor signal(s). The actuation system can be an actuation element.

Preferably one or more of the actuation sensors of the plurality of actuation sensors detect a specific kind of actuation, since the sensors are preferably responsive to the movement of the actuation system, and trigger(s) the actuation or calibration mode. For further explanations, it is referred for example to the first aspect described above.

The method preferably further comprises actuating the actuation system by at least one of the following: sensing a motion, touching and applying a force to the actuation system.

Preferably, the method comprises actuating the actuation system for a first predetermined period of time or by a first number of consecutive actuations to select the calibration mode.

Preferably, the method comprises actuating the actuation system for a second predetermined period of time or by a second number of consecutive actuations to select the actuation mode.

In an embodiment, the method comprises operating the control device in the calibration mode and the actuation mode simultaneously or alternately.

In an embodiment of the invention, the method further comprises detecting any motion of the control device with respect to a supporting surface.

In an embodiment of the invention, the method further comprises visually indicating to a user whether the control device is operating in the calibration mode or the actuation mode and/or visually indicating to a user the different functions assigned to the actuation system.

In a further embodiment, the visually indication is performed by a plurality of LEDs preferably corresponding in number and position to the plurality of actuation sensors.

The above objects are achieved according to a third aspect of the invention by a control device for controlling at least two different functions of an instrument or of at least one instrument or device in communication with the control device, the control device comprising:

a base element provided with a plurality of actuation sensors situated on the base element and preferably symmetrically distributed about a central axis,

an actuation element movably supported above the base element and movable toward the base element when a force is applied to the actuation element,

said actuation element being supported above the base element so as to be actuatable by movement in at least two different ways,

said plurality of actuation sensors situated on the base element so that at least one of said actuation sensors detects movement of the actuation element toward the base element,

said control device being operable in a calibration mode and in an actuation mode,

such that in the calibration mode a relative position between the control device and a user is defined and depending on said relative position, the at least two different functions are assigned to the at least two different ways of actuating the actuation element, and

such that in the actuation mode an actuation of the actuation element actuates an assigned function.

Preferably, in the actuation mode an actuation of the actuation element of the calibrated control device in either one of the at least two different ways of actuating the actuation element actuates or triggers the function assigned to the respective way of actuating the actuation element.

The above objects are achieved according to a fourth aspect of the present invention by a control device for controlling at least two different functions of an instrument or of at least one instrument or device in communication with the control device, the control device being operable in a calibration mode and in an actuation mode, the control device comprising:

an actuation system for actuating or operating the control device,

a sensor system for detecting an actuation of the actuation system,

wherein in the calibration mode, a relative position between the control device and a user is defined, and

depending on the relative position between the control device and the user,
preferably different or at least two different actuation areas are defined on the actuation system, and
the at least two different functions are assigned to (the) different actuation areas or to (the) at least two different actuation areas of the actuation system,

wherein in the actuation mode, the at least two actuation areas are actuatable to control the at least two different functions.

The actuation system can comprise the or one actuation element or a group of actuation elements, that is one or more actuation elements.

The above objects are achieved according to a fifth aspect of the present invention by a control device for controlling at least two different functions of an instrument in communication with the control device, the control device comprising an actuation element configured to be actuated in at least two different ways, wherein the control device is configured to be operated in a calibration mode and an actuation mode such that in the calibration mode a respective function of the at least two functions can be assigned to the at least two different ways of actuating the actuation element and such that in the actuation mode an actuation of the actuation element of the calibrated control device in either one of the at least two different ways of actuating the actuation element actuates or triggers the function assigned to the respective way of actuating the actuation element.

Preferably, in the calibration mode the control device determines its relative (angular) position relative to the user.

Preferably, in the calibration mode the assignment of a respective function of the at least two functions to the at least two different ways of actuating the actuation element is effected by an actuation of the actuation element, that is, by the actuation element being actuated.

Preferably, in the calibration mode the assignment of a respective function of the at least two functions to the at least two different ways of actuating the actuation element is effected by positioning a user's foot relative to the control device.

In an embodiment of the invention, the control device further comprises a base element floatingly supporting the actuation element.

In an embodiment of the invention, a cavity is defined between the base element and the actuation element and a plurality of actuation sensors are preferably arranged within the cavity, wherein the plurality of actuation sensors are configured to detect an actuation of the actuation element resulting in a motion of the actuation element towards the base element.

In an embodiment of the invention, the actuation element is floatingly supported by at least one support element, such as a spring element, preferably disposed on the base element.

In an embodiment of the invention, the base element substantially has the shape of a flat circle and/or the actuation element substantially has the shape of a radially symmetric plate turned upside down. The actuation element thus has preferably the shape of a dome.

In an embodiment of the invention, the control device is connected via a cable and/or wirelessly to the instrument in communication with the control device.

In an embodiment of the invention, the actuation element comprises or defines an actuation surface configured such that the at least two different ways of actuating the actuation element comprise the exertion of a force onto the actuation element at two different locations of the actuation surface.

In an embodiment of the invention, the control device is configured to operate in the calibration mode and the actuation mode simultaneously or alternately.

In an embodiment of the invention, the control device further comprises means for detecting any motion of the control device with respect to a supporting surface, preferably comprising a mouse ball, preferably configured to be in rolling contact with the floor surface.

In an embodiment of the invention, the control device further comprises means for visually indicating to a user whether the control device is operating in the calibration mode or the actuation mode and/or means for visually indicating to a user the different functions assigned to the actuation element. The means can be formed as an indication device or devices.

In an embodiment of the invention, the visual indication means comprise a plurality of LEDs preferably corresponding in number and position to the plurality of actuation sensors.

In an embodiment, the control device comprises a power supply or source or is connectable with or to a power supply or source. The power supply or source can also be for example a battery. Also the instrument or device can be powered via a power supply and/or can be connected to a power supply and/or can comprise a power supply or source.

The above objects are achieved according to a sixth aspect of the present invention by a control device for controlling at least two different functions of an instrument or of at least one instrument in communication with the control device, the control device being operable in a calibration mode and in an actuation mode, the control device comprising:

a base element,

an actuation element supported by and/or above the base element, and

a plurality of actuation sensors distributed on the base element about a central axis,

the plurality of actuation sensors being responsive to movement of the actuation element.

In an embodiment, the control device is configured or operable, such that in the calibration mode,

a relative position between the control device and a user is defined,

depending on the relative position between the control device and a user, the at least two different functions are assigned to at least two different actuation areas of the actuation element, and

such that in the actuation mode, the at least two actuation areas are actuatable to control the at least two different functions.

In an embodiment of the invention, the control device is configured such that, depending on the relative position between the control device and the user, the at least two actuation areas, which preferably form the actuation element, are definable.

In an embodiment of the invention, the base element is a planar base element. Preferably, the base element substantially has the shape of a flat disc or circle. Advantageously, the actuation element substantially has the shape of a radially symmetric plate turned upside down or has the shape of a dome.

In an embodiment of the invention, the plurality of actuation sensors are symmetrically distributed on the base element about the central axis. The central axis is preferably a notional line arranged in the center of the control device and arranged perpendicularly to the base element. The central axis can be a central symmetry axis or symmetry axis.

In an embodiment of the invention, the actuation element is floatingly supported by the base element.

In an embodiment of the invention, the actuation element is actuatable to select at least one of the following: the calibration mode and the actuation mode.

In another embodiment, the control device comprises a controller, for example a computer, a computer unit, a processor and/or a corresponding software, configured to perform at least one of the following: to process one or more sensor signals of or from the plurality of actuation sensors, to generate one or more function or functional signals and to transfer the one or more function signals to the instrument or to the at least one instrument or device or to a controller of the instrument or device, as for example exemplarily described below.

In another embodiment, the control device is configured to select a predefined assignment pattern from a plurality of predefined assignment patterns in order to define the at least two actuation areas of the actuation element. The control device may also work with only one predefined assignment pattern or one or more predefined assignment patterns.

In an embodiment of the invention, a cavity is defined between the base element and the actuation element and the plurality of actuation sensors are arranged within the cavity. The plurality of actuation sensors are preferably configured to detect an actuation of the actuation element, wherein the actuation of the actuation element results in a motion of the actuation element towards the base element.

In an embodiment of the invention, the actuation element is supported above the plurality of actuation sensors and actuatable by being movable toward the plurality of actuation sensors in response to an external force applied to the actuation element.

In an embodiment of the invention, the actuation element is supported, preferably floatingly supported, above the plurality of actuation sensors by at least one support element or a plurality of support elements.

According to a further embodiment, the actuation element is floatingly supported by and/or above the base element by at least one support element or by a plurality of support elements.

In a further embodiment, the at least one or the plurality of support elements are spring-biased, telescoping posts, which are arranged between the base element and the actuation element. In a further preferred embodiment, the at least one support element or the plurality of support elements is a spring element or are spring elements.

The control device can be connected via a cable and/or wirelessly to the at least one instrument in communication with the control device.

The control device can be configured to operate in the calibration mode and the actuation mode simultaneously or alternately.

Preferably, the control device further comprises a detection device or means for detecting any motion of the control device with respect to a supporting surface, preferably comprising a mouse ball configured to be in rolling contact with the floor surface.

According to a further embodiment the control device further comprises an indication device or means for visually indicating to a user whether the control device is operating in the calibration mode or the control mode and/or an indication device or means for visually indicating to a user the different functions assigned to the actuation element. Preferably, the visual indication means comprise a plurality of LEDs preferably corresponding in number and position to the plurality of actuation sensors. The indication device can be for example a display device. The display device is for example displaying or indicating the relevant information, for example also the position of the selected predefined assignment pattern.

In a further embodiment, the actuation system is actuatable in a respective way of at least two different ways to select at least one of the following: the calibration mode and the actuation mode.

A first way of the at least two different ways of actuating the actuation system is preferably an actuation for a first predetermined period of time or by a first number of consecutive actuations to select the calibration mode. A second way of the at least two different ways of actuating the actuation system is preferably an actuation for a second predetermined period of time or by a second number of consecutive actuations to select the actuation mode.

In a further embodiment, the actuation system is actuatable in a respective way of at least two different ways to select at least one of the at least two different functions.

In a further embodiment, each of the plurality of actuation sensors is in communicative relationship with the actuation element, for example with at least one actuation area of the at least two actuation areas, and wherein each actuation sensor of the plurality of actuation sensors is configured to detect an actuation of the actuation system. With the detection of an actuation, the calibration mode and/or the actuation mode and the corresponding steps can preferably be triggered.

According to a further embodiment, the control device is configured to automatically switch between the calibration mode and the actuation mode.

In a further embodiment, the control device comprises an indication device for visually indicating to a user a home position and/or a reference position of the control device. The home and/or reference position is preferably a starting point for the calibration mode. Preferably, a load point L1 represents the relative position of the user.

In an embodiment, the control device comprises a power supply or is connectable with or to a power supply. The power supply can be for example a battery.

The embodiments described with a specific aspect of the invention are also applicable to the other aspects, provided that a corresponding embodiment is in line with the corresponding aspect.

Additional advantages and features of the present invention are defined in the additional dependent claims and/or will become apparent by reference to the following detailed description and accompanying drawings.

The terms “instrument” or “device” may include instruments, devices, units or systems, for example an integral dental unit or a dental control center. The term “function” includes control functions, actuation functions, functions for controlling software etc.

### BRIEF DESCRIPTION OF THE DRAWINGS

FIG. 1 shows a top sectional view of an embodiment of a control device embodying the present invention.

FIG. 2 shows a cross-sectional view of the embodiment of the control device along the plane II-II of FIG. 1.

FIG. 3 shows a schematic representation of the embodiment of FIG. 1 and FIG. 2 with information concerning the operation of the control device.

FIGS. 4a to 4d show different assignment patterns, applicable for example in the embodiment shown in FIG. 1 and FIG. 2.

FIG. 5a shows the assignment pattern of FIG. 4c.

FIG. 5b shows a schematic representation of the embodiment of FIG. 1 and FIG. 2 with the assignment pattern of FIG. 5a assigned to the control device.

FIG. 6 shows a schematic representation of the embodiment of FIG. 1 and FIG. 2 with the assignment pattern of FIG. 5a assigned to the control device and with information concerning the operation of the control device.

FIG. 7 shows the top view of the control device of FIG. 1, with three different foot positions.

FIG. 8 shows a cross-sectional view of a further embodiment of a control device embodying the present invention.

FIG. 9 shows a top view of a further embodiment of a control device.

FIG. 10 shows a top view of a further embodiment of a control device.

FIG. 11 shows a diagram depicting signals reflecting the actuation of actuation sensors.

FIG. 12 shows a diagram depicting signals reflecting the actuation of actuation sensors plotted over time.

FIG. 13 is a representation showing an exemplary signal sequence for performing functions of an instrument in communication with a control device of the present invention.

FIG. 14 is a representation showing an exemplary signal sequence for performing functions of an instrument or device in communication with a control device of the present invention.

### DETAILED DESCRIPTION OF PREFERRED EMBODIMENTS

The present invention will now be further described by defining different aspects of the invention generally outlined above in more detail. Each aspect so defined may be combined with any other aspect or aspects unless clearly indicated to the contrary. In particular, any feature indicated as being preferred or advantageous may be combined with any other feature or features indicated as being preferred or advantageous.

The control device can be formed as a foot-actuated or foot-actuatable device. However, the control device can also be formed or provided as a hand-actuated or hand-actuatable device. In other words, the control device can be formed as a foot- or hand-operated device. Preferably, the control device of this invention is configured as a foot control device or a hand control device.

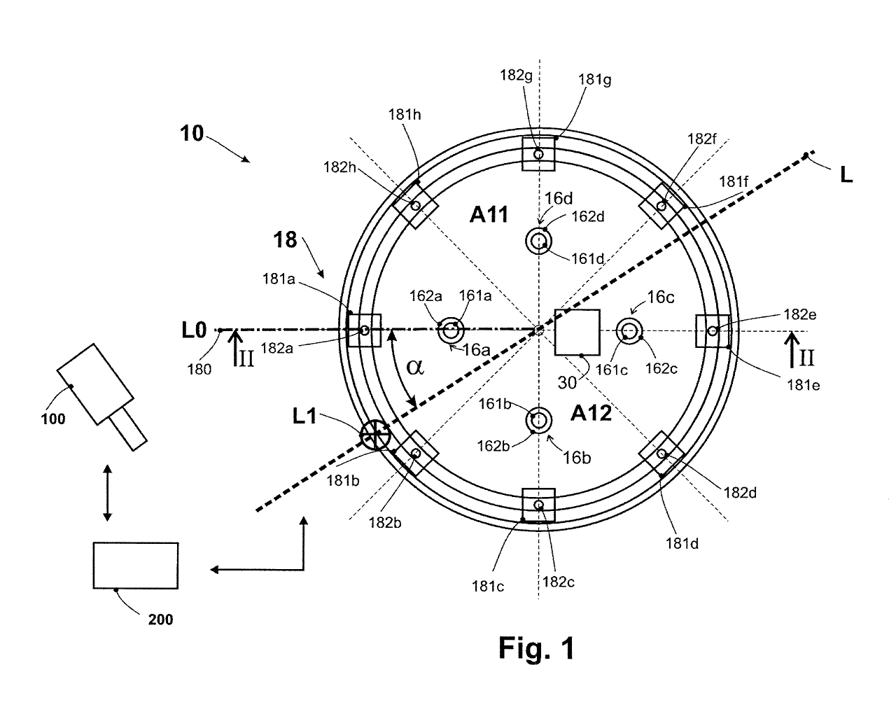

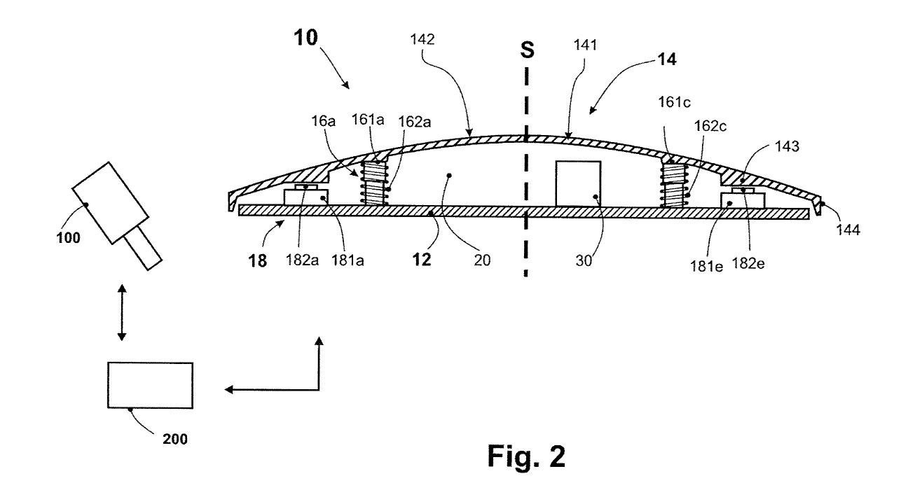

An embodiment of a control device 10 embodying the present invention is schematically shown in FIGS. 1 and 2. FIG. 1 shows a top sectional view of an embodiment of a control device embodying the present invention. FIG. 2 shows a cross-sectional view of the embodiment of the control device along the plane II-II of FIG. 1. The components inside the control device are not illustrated in cross-sectional view. The control device 10, which in particular can be operated by the foot or also the hand of a user, for controlling an instrument 100, for example a medical or dental equipment or instruments 100 or any other kind of instrument, device or equipment, for example a musical instrument, comprises a base element 12, see FIG. 2, and an actuation element 141 of an actuation system 14, for example a convex contact plate or dome juxtaposed over the base element 12. The actuation element 141 is preferably provided with a peripheral shoulder 143, see FIG. 2, which defines a contact surface for a sensor system 18. The sensor system 18 comprises in this embodiment a plurality of actuation sensors 181a, 181b, 181c, 181d, 181e, 181f, 181g, 181h, such as for example switch posts. In this embodiment, the actuation system is the actuation element.

Preferably shoulder 143 is unitary with the actuation element 141 or may be formed as a single element. As can be seen from the plan sectional view shown in FIG. 1, the control device 10 in this embodiment has a substantially circular, radially symmetric shape relative to a central axis S shown in FIG. 2. The base element 12 has substantially the shape of a flat disc.

As shown in FIG. 2, the top surface of the actuation element or actuation system or contact plate 141 defines an actuation surface 142 for operator or user contact. The actuation element 141 comprises at least two different actuation areas (see FIG. 1), for example actuation area A11 and actuation area A12 which can be for example defined during operation of the control device as explained in more detail below. That is, the actuation system 14 comprises the actuation element 141. The actuation areas A11 and A12 form the actuation element 141.

The control device comprises in this embodiment a power supply 30 or is connectable with or to a power supply. The power supply can be for example a battery. Also the instrument or device can be powered via a power supply.

Optionally, the bottom surface of the base element 12 can be outfitted with a non-skid, rubber backing to facilitate keeping the base element 12 and, thus, the control device 10 in place on a support surface, such as the floor or the like.

The base element and the actuation element are preferably connected by means of support elements, e.g. spring elements, such that the actuation element is floatingly supported by the base element. That is, the actuation element 141 is preferably supported by and/or above (in use) the base element 12 by support elements 16a, 16b, 16c, 16d, such as for example four, uniformly spaced telescoping and spring-biased posts. Thus, the actuation element 141 is floatingly supported above or by the base element 12. In this embodiment, the support elements are formed by telescoping or support posts 161a, 161b, 161c, 161d and spring elements 162a, 162b, 162c, 162d, which are arranged between the base element and the actuation element. In this floating configuration the actuation element 141 can be urged or moved towards the base element 12, for instance, by exerting an external force F on the actuation surface 142 and to at least one of the actuation areas A11, A12, respectively, of the actuation element 141. Deformation or partial collapse of some or all of the support elements 16a-16d, depending on the exact position of the force exerted by the user on the actuation surface 142, causes a portion of the actuation element 141, e.g., peripheral shoulder 143, to approach or contact one or more actuation sensors 181a-181h. Once this force is no longer being exerted, the actuation element 141 will return to its default position due to the restoring force exerted by the biased spring elements 162a-162d of or arranged around the support posts 161a-161d. As shown in FIG. 1, for example four support elements 16a-16d are distributed symmetrically around the central axis S of the control device 10.

A person skilled in the art will appreciate that different arrangements of support elements as well as a different number of support elements are also possible without departing from the present invention, for example only one support element, for example arranged along the central axis S. Moreover, the person skilled in the art will appreciate that a bias element other than springs or spring elements can be used with the support posts as well. The at least one support element or the plurality of support elements can also be formed on the basis of a mere spring element or elements or on the basis of a foamed material.

As already indicated above, the actuation element 141 of this embodiment is formed of or includes two actuation areas A11, A12 once calibrated.

As shown in FIG. 2, the actuation element 141 together with the base element 12 and the support elements 16a-16d define a cavity 20 between the base element 12 and the actuation element 141 and actuation areas A11, 12 of the actuation system 14, respectively. That is, due to the preferred floating support of the actuation element by the base element and the support elements a cavity is defined between the base element and the actuation element. In this embodiment, the sensor system 18 comprising the plurality of the actuation sensors 181a-181h, such as for example switch posts, is arranged within the cavity 20. The sensor system 18 is preferably in communicative relationship with the actuation system 14. The actuation sensors are configured to detect or sense any actuation, in particular movement of the actuation system 14 and thus, for example of the actuation element 141 towards the base element 12.

Preferably, the actuation sensors are disposed on the base element 12 near the peripheral edge thereof. Preferably, the plurality of actuation sensors are distributed on the base element about the central axis S. Preferably, the plurality of actuation sensors are symmetrically distributed on the base element about or around the central axis. Preferably, eight actuation sensors 181a-181h are symmetrically disposed on the base element 12 about the central axis S near the peripheral edge thereof and spaced relative to one another by about 45 degrees of an arc. In this embodiment, each actuation sensor 181a-181h comprises a retractable or movable pin, such as pins 182a, 182b, 182c, 182d, 182e, 182f, 182g, 182h, that is biased, for instance, by means of an internal spring element into an extended position. In this extended position, the tip of the pin of each actuation sensor 181a-181h is in relatively close proximity, or almost abutting relationship, with a bottom surface of the actuation element 141, preferably with the peripheral shoulder 143 of the actuation element 141. For instance, in the embodiment shown in FIG. 2, the tip of each pin of each actuation sensor 181a-181h almost abuts the annular ledge or peripheral shoulder 143 being defined by the bottom surface of the actuation element 141. The movable pin(s) 182a-182h can also be a pushbutton switch. The actuation sensors 181a-181h can also provide a proximity switch.

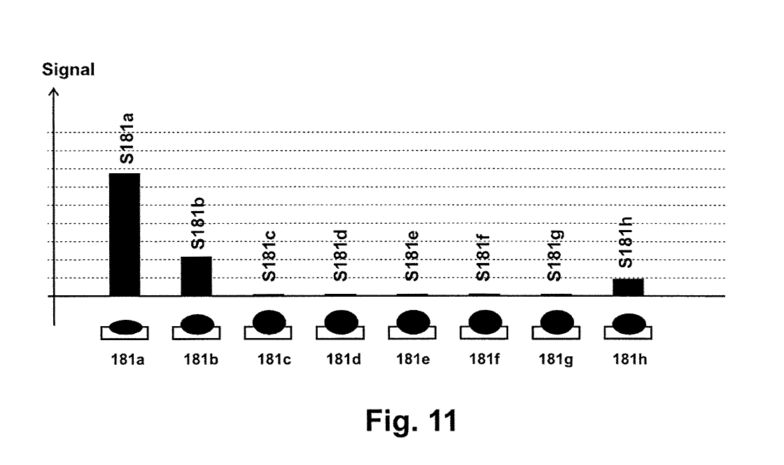

Upon depression of the actuation element 141 by a user, for example by a user exerting a force on the actuation surface of the actuation element, some or all of the pins of the actuation sensors 181a-181h, because of their abutting relationship with the bottom surface of the actuation element, will be displaced from their normally extended position(s) towards their retracted position(s). Thus, downwardly motion of the actuation element 141 will be sensed or registered by at least one, some, or all of the actuation sensors 181a-181h. Actuation or activation of the actuation sensors 181a-181h in this manner will preferably generate or trigger corresponding actuation signal(s) or sensor signal(s) S181a, S181b, S181c, S181d, S181e, S181f, S181g, S181h, see for example FIG. 11. The signal(s) initially can trigger the calibration mode and/or the actuation mode in order to perform the corresponding steps. In the actuation mode, the signal(s) can be communicated for example to an instrument 100 in communication with the control device 10. The actuation signal(s) or sensor signal(s) can also be communicated to an internal and/or external controller 200 or computer, computer unit or processor of the control device 10 for processing the sensor signals S181a-S181h from the actuation sensors 181a-181h and communicating a resulting function or control signal SF or resulting function or control signals SF to the instrument 100 in communication with the control device 10. In other words, due to the actuation of the actuation system 14, in this embodiment for example of the actuation element 141 and, in the actuation mode, of at least one of the actuation areas A11, A12 (see for example FIG. 1), one or more of the actuation sensors 181a-181h are actuated in order to generate one or more corresponding signals S181a-S181h. The one or more signals allow for triggering one or more desired steps, which is described below in more detail.

The person skilled in the art will appreciate that the number, configuration and arrangement of the above described actuation sensors 181a-181h corresponds to a preferred embodiment and a different number of differently arranged actuation sensors having a different configuration can be used as well. For instance, the actuation sensors can be configured to sense the magnetic fields produced by magnetic elements disposed in or on the actuation element so that an actuation of the actuation element will result in a different magnetic field sensed by the actuation sensors and the triggering of a corresponding actuation signal.

In this embodiment, the sensor system 18 comprises a plurality of actuation sensors 181a-181h, wherein each of the plurality of actuation sensors is in communicative relationship (or in communication) with at least one actuation area of the at least two actuation areas, preferably upon actuation of the actuation element. Each of the plurality of actuation sensors is configured to detect an actuation of the actuation system 14. In a further embodiment, only one actuation sensor can be provided, for example a weighing sensor which works like a wobble plate or wobble disc. Such a type of sensor is preferably arranged along the central axis S, in this embodiment for example between the base element 12 and the actuation system 14.

The actuation element 141 can also be provided with a circumferential, annular skirt 144 for preventing the accumulation of any dust or dirt within the cavity defined between the base element 12 and the actuation element 141.

The control device 10 is configured to operate in two modes, namely a calibration or gauge mode on the one hand and an actuation or control mode on the other hand. The person skilled in the art will appreciate from the following detailed description that the details of these two different modes of operation can be implemented in the control device 10 in a number of different ways.

In the calibration or gauge mode the control device 10 is preferably configured to determine its relative, in particular its angular position with respect to the user and calibrate itself accordingly. Preferably, a relative position of the control device 10 to a user is defined or is definable or a relative position between the control device and a user is defined, and, depending on the relative position between the control device and the user, at least two different functions are assigned to at least two different actuation areas of the actuation system and actuation element, respectively. Preferably, the assignment is performed so that not more than one function is assigned to one actuation area. Any double assignment or assignment of two functions to one actuation area can be avoided if desired.

Assigning a function to a certain actuation area preferably results in an assignment of said function to one or more sensors. “Assigning a function to an actuation area” preferably also means “assigning an actuation area to a function”, and “assigning a function to a sensor (to one or more sensors)” preferably means “assigning a sensor (one or more sensors) to a function”. The control device can preferably be configured so that any double assignment, that is, more than one function is assigned to one actuation area, is avoided. Furthermore, assigning functions to certain actuation areas can preferably also be seen as an assignment of the at least two functions to at least two different ways of actuating the actuation element.

According to certain embodiments of the present invention the control device 10 can be configured to change from the calibration or gauge mode to the actuation or control mode for example by an appropriate actuation of the actuation system 14, in this embodiment for example of the actuation element 141. Alternatively, the control device 10 can preferably operate in both modes simultaneously, wherein a calibration of the control device 10, that is, selecting the calibration mode, is achieved by a first way of actuating the actuation element 14, such as by depressing the actuation element 141 for a relatively longer time period, a first predetermined time period, e.g., more than 5 seconds, and an actuation of the control device 10, that is, selecting the actuation mode, is achieved by a second different way of actuating the actuation element 14, such as by depressing the actuation element 141 for a relatively shorter time period, a second predetermined time period, e.g., less than 5 seconds. In further embodiments, other types of triggering or selecting the calibration mode and the actuation mode, respectively, can be provided. For example, a first number of consecutive actuations as a first way of actuating the actuation element or actuation system can be provided to select the calibration mode, and a second number of consecutive actuations as a second way of actuating the actuation element or actuation system can be provided to select the actuation mode. In a further embodiment, a further way of actuating the actuation system is positioning a user's foot for example in a specific manner relative to the control device.

The actuation system is preferably actuatable by at least one of the following: sensing a motion, touching and in particular applying a force to the actuation system in order to trigger or select the calibration mode and/or the actuation mode.

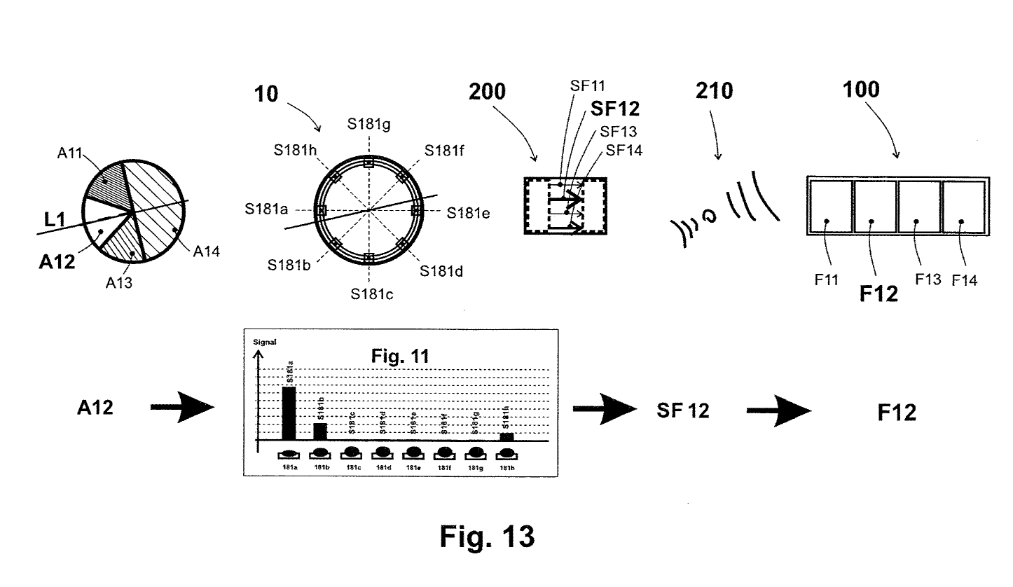

One preferred embodiment of working with the calibration mode and the actuation mode will now be described in the context of FIGS. 1 and 2. Preferably, in the actuation or control mode the control device 10 acts as a multifunctional switch or multi-function control device, i.e., a switch that can control or trigger multiple functions, for example functions F11, F12, . . . (see for example FIG. 13) of an instrument or of more than one instrument in communication with the control device 10, wherein the different functions of the instrument(s) in communication with the control device 10 are assigned to individual actuation areas (in this embodiment for example actuation areas A11, A12) and thus, to individual actuation sensors 181a-181h or groups of adjacent actuation sensors 181a-181h so that an actuation of the actuation element 141 via its actuation surface 142 at or close to the position of a certain actuation sensor 181a-181h will trigger the function assigned to this actuation sensor or the group of actuation sensors this actuation sensor belongs to. For example, in the preferred embodiment shown in FIGS. 1 and 2 four of the eight actuation sensors 181a-181h, such as the actuation sensors 181a-181d, could be assigned to a first function (for example rinsing), and the other four of the eight actuation sensors, such as the actuation sensors 181e-181h, could be assigned to a second function (for example cutting) of an instrument in communication with and to be controlled by the control device 10. In other words, an actuation or activation of at least one, some or all of the pins of the actuation sensors 181a-181d by a user exerting a force on the actuation surface 142 of the actuation element 141 somewhere in the semicircular region defined by the actuation sensors 181a-181d will trigger the first function assigned to these actuation sensors 181a-181d. Likewise, an actuation or activation of at least one, some or all of the pins of the actuation sensors 181e-181h by a user exerting a force on the actuation surface 142 somewhere in the semicircular region defined by the actuation sensors 181e-181h will trigger the second function assigned to these actuation sensors 181e-181h. As described above, the first and the second function could relate to two different functions of one instrument controlled by the control device 10. Alternatively, the first and the second function could relate to the operation of a first instrument and a second instrument in communication with and controlled by the control device 10.

Preferably, in the calibration or gauge mode the control device 10 is configured to determine its relative (angular) position with respect to the user and assign specific (control) functions to respective ones of the actuation sensors 181a-181h. Such an assignment between the actuation sensors 181a-181h and the specific functions to be controlled by the control device 10 requires the interaction with the user. Preferably, in the calibration or gauge mode of the control device 10 a depression of the actuation element 141, that is, for example the depression of one part or area of the actuation element, towards the base element 12 by the user's foot, hand or finger applying a force onto the actuation surface 142 near the peripheral edge thereof will lead to such an assignment between the actuation sensors 181a-181h and the specific functions, i.e., to a calibration of the control device 10, according to the preferred calibration mechanism described below. Alternatively, the control device 10 can comprise additional sensors for detecting the position and/or motion of the user's foot such that only by means of the position of the user's foot, i.e., the location of the user's foot above the actuation surface 142 of the actuation element 141, the control device 10 can be calibrated (i.e., its relative position to the user's foot can be for example determined and the specific control functions can be assigned to the actuation sensors 181a-181h accordingly).

That is, in a preferred embodiment, in the calibration mode the assignment of a respective function of the at least two functions to a corresponding actuation area is effected by an actuation of the actuation element. In a further embodiment, in the calibration mode the assignment of a respective function of the at least two functions to a corresponding actuation area is effected by positioning a user's foot relative to the control device.

The control device 10 can also act as a dual function switch, i.e., a switch that can control for example at least two different functions of an instrument in communication with the control device 10. When the user exerts a force onto the actuation surface 142, near a peripheral edge thereof, at a position that in the plan view of FIG. 1 lies between two actuation sensors, for example between the actuation sensors 181a and 181b, such as at the exemplary position L1, in this case a load point or load position which, in the calibration mode, defines or is a reference position or calibration value, indicated in FIG. 1 by a cross, the actuation sensors 181a-181h will be assigned to different instrument functions or will be calibrated as follows. The actuation sensors lying on one side of a notional line L or load line L running in the plan view of FIG. 1 from the point L1 or reference position through the center of the control device 10 will be assigned to a first function of the instrument in communication with the control device 10, whereas the actuation sensors lying on the other side of this notional line L will be assigned to a second function thereof. For example, in the embodiment shown in FIG. 1 the actuation sensors 181a and 181f-181h will be assigned to the first function of the instrument in communication with the control device 10, whereas the actuation sensors 181b-181e will be assigned to the second function thereof. In other words, the corresponding functions will be assigned to the corresponding actuation areas A11, A12 and actuation sensor(s), respectively. In the rather unlikely case that a user will exert a force directly above an actuation sensor so that the notional line L in the exemplary embodiment of FIG. 1 runs through two actuation sensors, one of these actuation sensors can be assigned to the first function of the instrument in communication with the control device 10 and the other one can be assigned to the second function thereof.

The control device is preferably provided with a zero or home position indicated by line L0. The home position can be seen as a first reference position. Once a new load point L1 has been defined or determined in the calibration mode, a second or new reference position is provided, which preferably serves as a reference position for all subsequent actuations during the actuation mode. That is, in the calibration mode, the load point L1 is determined and thus, determined or defined as a reference position, and in or during the actuation mode, further load points—the number of such further load points can be high during operation of the control device in the actuation mode—determine or define the actuation positions. On the basis of the further load points, the corresponding functions of the instrument or device are performed. In a subsequent calibration phase, a new reference position can be defined. The reference position is preferably a basis for assigning the at least two different functions to the at least two different actuation areas and is preferably a basis for one or more subsequent actuations of the actuation element during the actuation mode.

FIG. 1 shows a calibration angle α which is defined as the angle between the notional or load line L and the home position L0. The home position L0 is for example provided at the actuation sensor 181a.

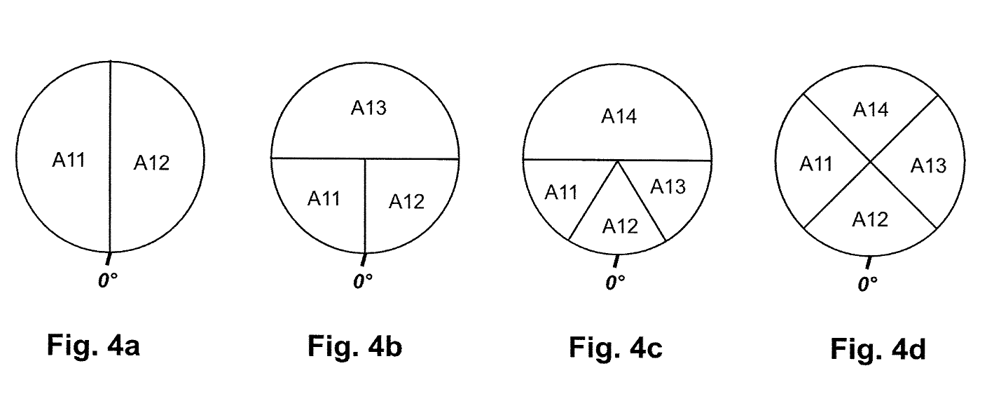

The person skilled in the art will appreciate that the radial symmetric arrangement of eight actuation sensors 181a-181h corresponds to a preferred exemplary embodiment. The present invention can also be implemented with more or less actuation sensors as well as with different arrangements of actuation sensors. It is contemplated, for instance, that arrangements of actuation sensors can be implemented according to the present invention where any notional line running in a plan view through one actuation sensor and the center of the control device does not run through another actuation sensor, as is the case in the embodiment shown in FIG. 1. Moreover, the person skilled in the art will appreciate that according to the present invention calibration mechanisms similar to the above can be used to assign more than two functions of an instrument in communication with the control device 10 to the actuation sensors 181a-181h thereof. For instance, it is possible to use a first notional line running through the point of exertion of force (reference position) and the center of the control device 10 as well as a second notional line that is perpendicular thereto and also runs through the center of the control device to define four quarter sections (see for example FIG. 4d), that is, four actuation areas, of the control device 10 corresponding to four different functions of an instrument to be controlled thereby.

Once the control device 10 has been calibrated, for instance by means of the above described preferred calibration mechanism, according to certain preferred embodiments, the control device 10 will no longer operate in the calibration or gauge mode but in the control or actuation mode. For instance, in a control device 10 that has been calibrated as indicated in FIG. 1, i.e., by exerting a force onto the actuation surface 142 at the position L1 and thereby assigning the actuation sensors 181a and 181f-181h to a first function of an instrument in communication with the control device 10 and the actuation sensors 181b-181e to a second function thereof, an exertion of a force on the actuation surface 142 at the actuation area A11 on the, from the user's point of view “left side” of the notional line L in the plan view of FIG. 1 will lead to a triggering of the first function of the instrument in communication with the control device 10, whereas an exertion of a force on the actuation surface 142 at the actuation area A12, on the “right side” of the notional line L in the plan view of FIG. 1 will lead to a triggering of the second function of the instrument in communication with the control device 10.

As already described above, according to alternative preferred embodiments of the present invention the control device 10 can operate in both modes, i.e., on the one hand the calibration or gauge mode and on the other hand the control or actuation mode, simultaneously, wherein a calibration of the control device 10 is achieved for example by means of a first way of actuating the actuation element 141 (for example contact plate), such as by depressing the actuation element 141 at any position of the actuation surface 142 for more than for example 5 seconds, that is for a first predetermined period of time, and an actuation of the control device 10 is achieved by means of a second different way of actuating the actuation surface 142 of the actuation element 141, such as by depressing the actuation element 141 (at actuation area A11 or actuation area A12) for less than for example 5 seconds, that is for a second predetermined period of time.

The person skilled in the art will appreciate that also in these alternative embodiments a calibration and actuation mechanism as described above can be implemented in the control device 10. For instance, exerting a force for more than 5 seconds at the position of the actuation surface 142 of the actuation element 141 marked L1 in FIG. 1 preferably results in an assignment of the actuation sensors lying on the “left side” of the notional line L running in the plan view of FIG. 1 from the point L1 through the center of the control device 10, i.e., the actuation sensors 181a and 181f-181h, to a first function of an instrument in communication with the control device 10 and an assignment of the actuation sensors lying on the “right side” of this notional line L, i.e., the actuation sensors 181b-181e, to a second function thereof. Having calibrated the control device 10 in such a way, an exertion of a force for less than for example 5 seconds on the actuation surface 142, that is on the actuation area A11, of the actuation element 141 on the “left side” of the notional line L in the plan view of FIG. 1 will lead to a triggering of the first function of the instrument in communication with the control device 10, whereas an exertion of a force for less than for example 5 seconds on the actuation surface 142, that is on the actuation area A12, of the actuation element 141 on the “right side” of the notional line L in the plan view of FIG. 1 will lead to a triggering of the second function of the instrument in communication with the control device 10. An exertion of a force for more than for example 5 seconds on the actuation surface 14a of the contact plate 141 will result in another calibration of the control device, for instance, according to the above described preferred calibration mechanism.

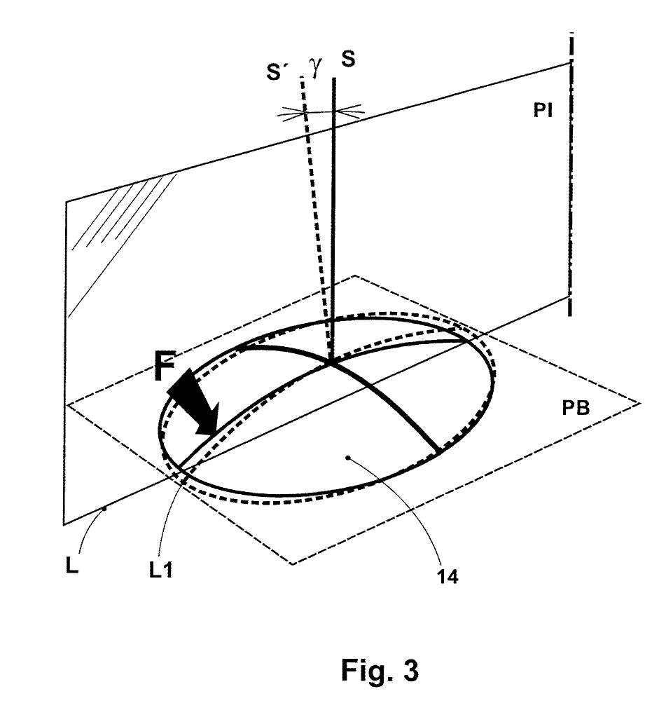

FIG. 3 shows a schematic representation of the embodiment of FIG. 1 and FIG. 2 with information concerning the operation of the control device or in other words: a method of operating the control device for controlling the at least two different functions of an instrument or at least one instrument in communication with the control device. FIG. 3 shows a possibility how the relative position between the control device 10 and the user is defined as a basis for assigning the at least two different functions to the at least two different ways of actuating the actuation element. This is for example achieved by exerting a force F to the actuation system 14, that is to the actuation element 141. The force F results in a subsequent determination and definition of the load point/reference position L1, the load direction and notional line or load line L (see for example also notional line L in FIG. 1). The actuation element 141 is, in this embodiment, as already described with for example FIGS. 1 and 2, provided as a radially symmetric convex plate or dome. The force F can be applied to said actuation element, for example by foot or hand.

In the following, one way of defining a reference position is explained as an example: As long as no force is applied to the actuation element, the control device is in a rest position. In a preferred case of a symmetrically designed device as shown in FIG. 1 the imaginary central axis S is preferably arranged in the center of the control device and arranged perpendicularly to the base element. Once, a force F is applied to the actuation element, the axis S changes its position or orientation, resulting in an inclined orientation in relation to the orientation in the rest position. That is, due to the load, the inclination of the actuation element 141 is created—in this embodiment, the actuation element, at least partly, moves towards the base element—and the axis is tilted which results in the inclined position, that is, inclined axis S′. An angle γ, inclination angle or load angle γ, is defined by axis S and inclined axis S′. The incline/inclination and the corresponding angle γ can be measured in the calibration mode; an inclination of the plate results in a measureable main direction of the inclination, see for example direction of the load line L in FIG. 1. Thus, a sensor signal results in an angular value, which represents the load point L1 in FIG. 1, that is, the (new) reference position is defined.

Geometrically, the incline of the actuation element 141 defines an inclination plane PI, wherein said inclination plane PI is defined by the axis S and the inclined axis S′. Said inclination plane PI intersects a base plane PB along a straight line L, that is notional line L, at an angle of 90°, see also FIG. 1. The load point L1 serves as a reference point or position for all subsequent actuations during the actuation mode.

In a further embodiment the definition of the load point/reference position L1 in the calibration mode is performed by the actuation system 18, comprising for example one or more weighing sensors, or a 2D inclination measuring device, for instance a 2D tilt sensor. The tilt sensor is preferably arranged or mounted between the actuation element and the base element in a central position. It is also possible to use a measuring device, which determines or detects the inclination angle in a different manner.

There exist several equivalent alternatives, how to determine the load point and the reference position, respectively and/or load direction and load line L in the calibration mode or subsequent load points during the actuation mode and during the operation of the control device. That is, the control device can operate with different types of actuation sensors:

array and plurality of sensors (actuation sensors), for example load cell(s) or distance rule(s) or distance scale(s),

2D inclination sensor as a tilt joint of, for example, the actuation element 141 or a 2D inclination measurement device (MEMS)

bubble level with automatic observation and/or inclination measurement,

one or more combined sensors, such as a level indicator or bubble level,

one or more electronic weighing sensors,

one or more electronic load sensors or load cells,

one or more electronic path or position sensors,

one or more capacitive and/or inductive sensors, and/or

combinations thereof.

The assignment of the functions can relate to one or more instruments or devices, for example a 3D graphics system. The functions assigned to the corresponding actuation areas can be at least one of the following: a zoom function, a “next” function, a shot image function etc. Also cameras or measurement devices, music instruments, tools etc. can be controlled by the described control device.

FIGS. 4a to 4d show different assignment patterns, that is, predefined assignment patterns, applicable for example in the embodiment shown in FIG. 1 and FIG. 2. A11 of the predefined assignment patterns show a home or 0° position for orientation and assignment purposes. A first arrangement or assignment pattern, see FIG. 4a, comprises the areas A11 and A12 as for example described with FIGS. 1 and 2. The area A11 is defined on the left side of notional line L and the area A12 is arranged on the right side of notional line L. A second different arrangement or assignment pattern, shown in FIG. 4b, comprises or has the areas A11, A12, A13. A third arrangement or assignment pattern, shown in FIG. 4c, comprises areas A11, A12, A13, A14, and a further different arrangement or assignment pattern, shown in FIG. 4d, comprises also areas A11, A12, A13, A14, however, arranged in a different manner. Further or different arrangements are of course possible. It should be noted that two, three, four, five, six or more actuation areas can be defined; in the embodiment shown in FIG. 1, two actuation areas, A11 and A12, are defined.

These different assignment patterns can be assigned to the actuation system 14, that is to the actuation element 141, in order to define the desired actuation areas during the calibration mode. In this way each actuation area of the two or more actuation areas is assigned to an explicit function of the two or more different functions, or the at least two different functions are assigned to the at least two different actuation areas.

The at least two actuation areas are preferably definable during the calibration mode. That is, the control device is configured such that the actuation areas can be defined and thus, redefined in subsequent calibration modes. In the case of the contact plate of the embodiment shown in FIGS. 1 and 2, the actuation areas are defined during the calibration mode. That is, the location of the actuation areas are defined during the calibration mode.

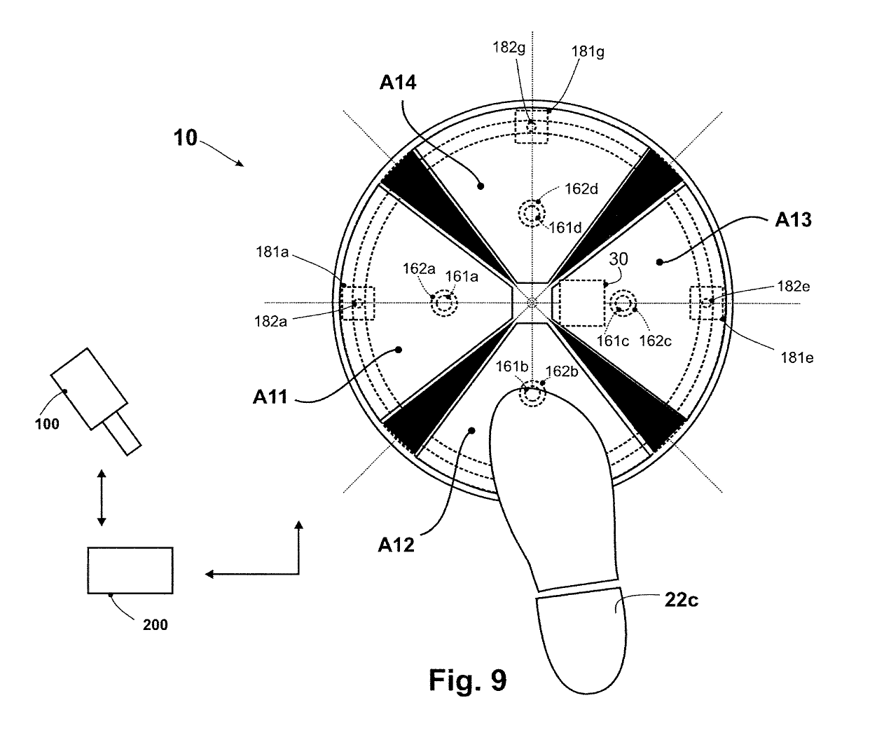

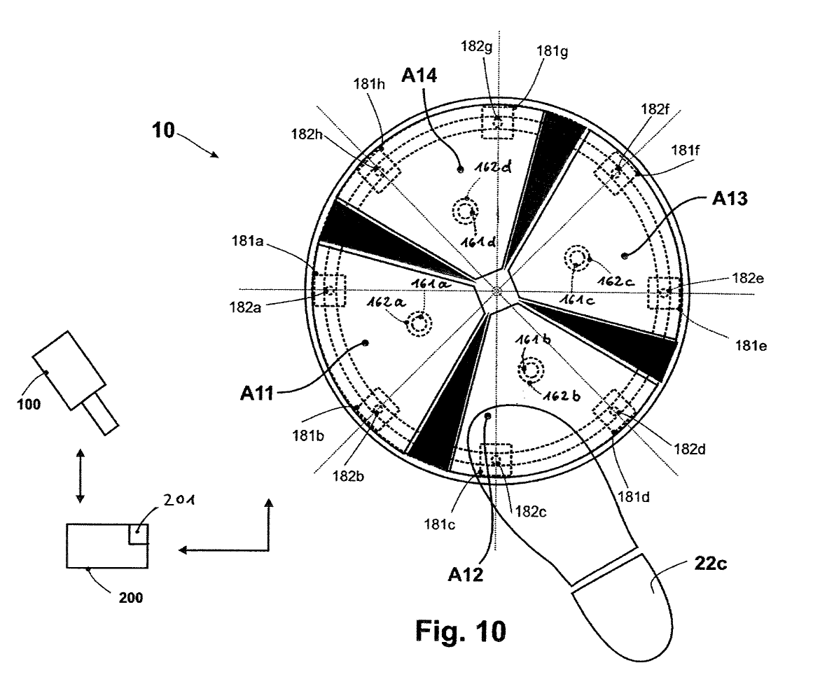

As can be taken for example from FIG. 9 or 10, the actuation areas are predefined, that is, their location is predefined or fixed. However, the assignment with a corresponding function can be changed or modified by starting a new calibration mode.

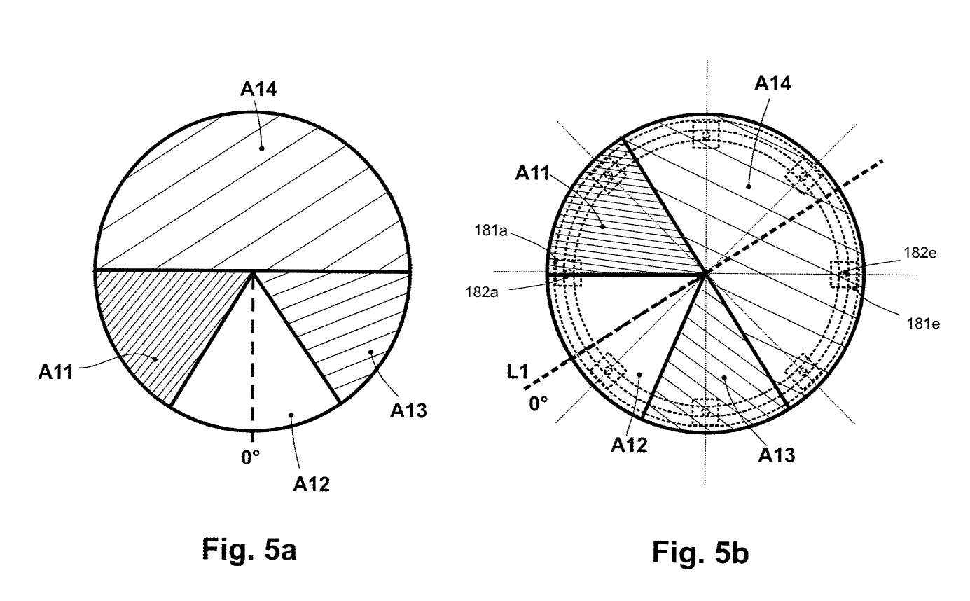

FIG. 5a shows the assignment pattern of FIG. 4c, wherein the different areas are marked in different manners. FIG. 5b shows a schematic representation of the embodiment of FIG. 1 and FIG. 2 with the assignment pattern of FIG. 5a assigned to the control device. A home position 0° of the assignment pattern shown in FIG. 4c or FIG. 5a is coincide with the load point L1 in FIG. 5b which is a reference position defined in the calibration mode. That is, the assignment pattern is aligned in respect of the load point L1 such that the home position or zero position of the predefined assignment pattern matches the load point L1. There may be several mechanisms for selecting a corresponding assignment pattern.

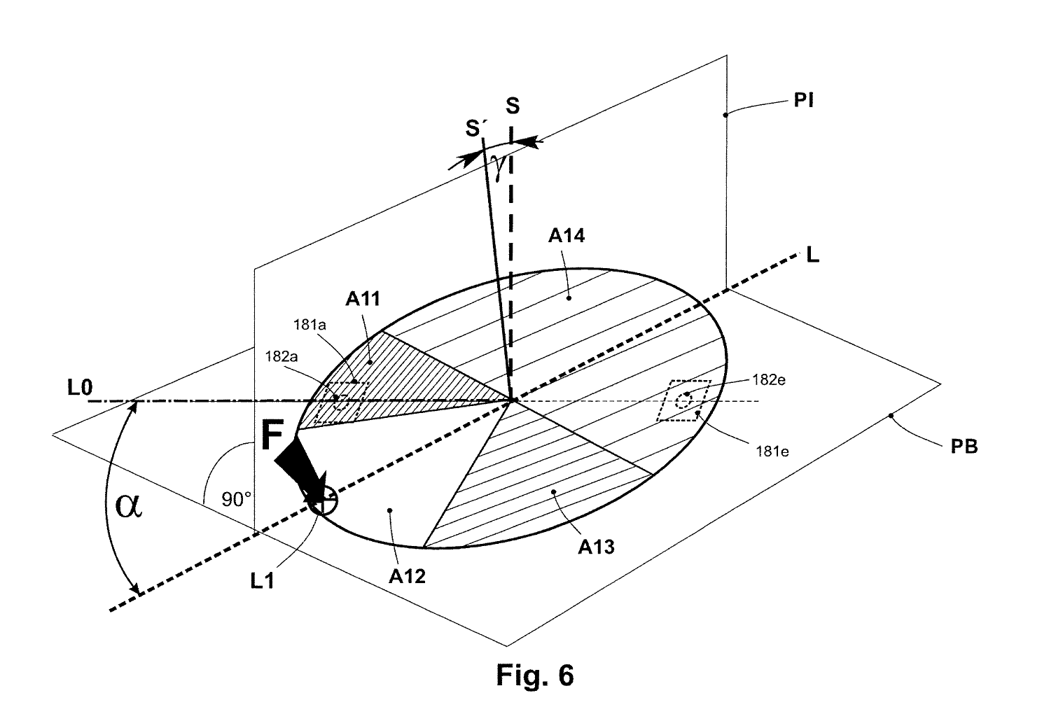

FIG. 6 shows a schematic representation of the embodiment of FIG. 1 and FIG. 2 with the assignment pattern of FIG. 5a assigned to the control device (assigned to the control device and actuation element, respectively, in a similar manner as shown in FIG. 5b) and with information concerning the operation of the control device. That is, information concerning a preferred method of operating the control device for controlling the at least two different functions of the instrument or at least one instrument in communication with the control device is shown. In particular, FIG. 6 shows the result of an exemplary process or method of assigning functions and actuation areas to each other, thereby using for example an assignment pattern as depicted in FIGS. 4c and 5a, respectively. As shown in FIG. 6, the home position of the assignment pattern is exactly positioned where the force F was applied during the calibration mode (reference position). This can be performed for example by an instruction, for example via the controller 200: put or place the home position (0° position) of the assignment pattern, see FIG. 4, on reference position or load point L1, see FIG. 6. Then, the actuation areas A11, A12, A13 and A14 are defined accordingly. Again, in this specific embodiment, said inclination plane PI intersects a base plane PB along a straight line L, that is notional line L, at an angle of 90°. The point L1 preferably serves as a reference point or position for all subsequent actuations during the actuation mode. Further load points, caused by a user during the actuation mode, in order to trigger corresponding functions, are interpreted by the controller 200 in relation to the reference point or reference position, which was defined during the calibration mode. For further explanations see for example FIG. 3.

It should be noted that the control device may be formed for example as a circle or as an ellipsoid or may be also formed in a linear manner or elongated. Also other arrangements are possible.

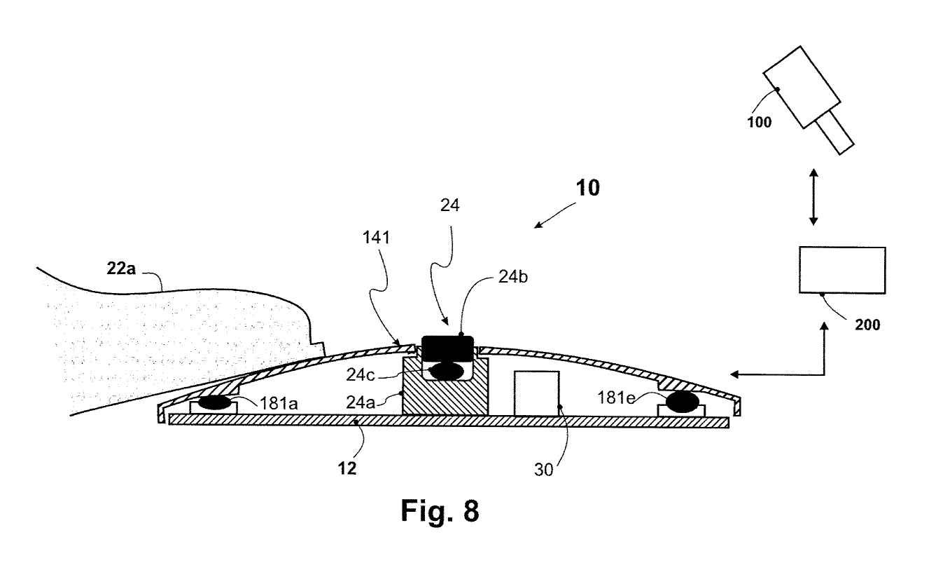

In a further embodiment, the user has the opportunity to select a new relative position between the user and the control device 10. In order to do so, a new calibration process has to be started. Starting a new calibration process can be performed by a predefined way of actuation, for example applying a force F for longer than for example 5 seconds or by pressing a predefined mode switch element or system 24 shown in FIG. 8.

In a further embodiment, and as already indicated above, the control device 10 is configured to operate with different assignment patterns as shown in FIG. 4a, 4b, 4c or 4d. The selection of a specific assignment pattern is performed by either a selection element (not shown) or the selection of the assignment pattern is determined by the selection of the controlled instrument. Other selection methods, such as voice control etc. are possible as well.

In the case that the control device is operated with an assignment pattern selected from a plurality of different assignment patterns, the user can change his or her relative position with respect to the control device and select the previously used or a new assignment pattern. The user can also maintain his or her position and select another assignment pattern. The control device can also work without the use of different assignment patterns. Merely the definition of a relative position between the control device and a user, for example by exerting a force onto the actuation element, that is, for example by determining and/or defining a reference position, can initiate an assignment of functions to separate actuation areas, in particular based on one predefined assignment pattern.

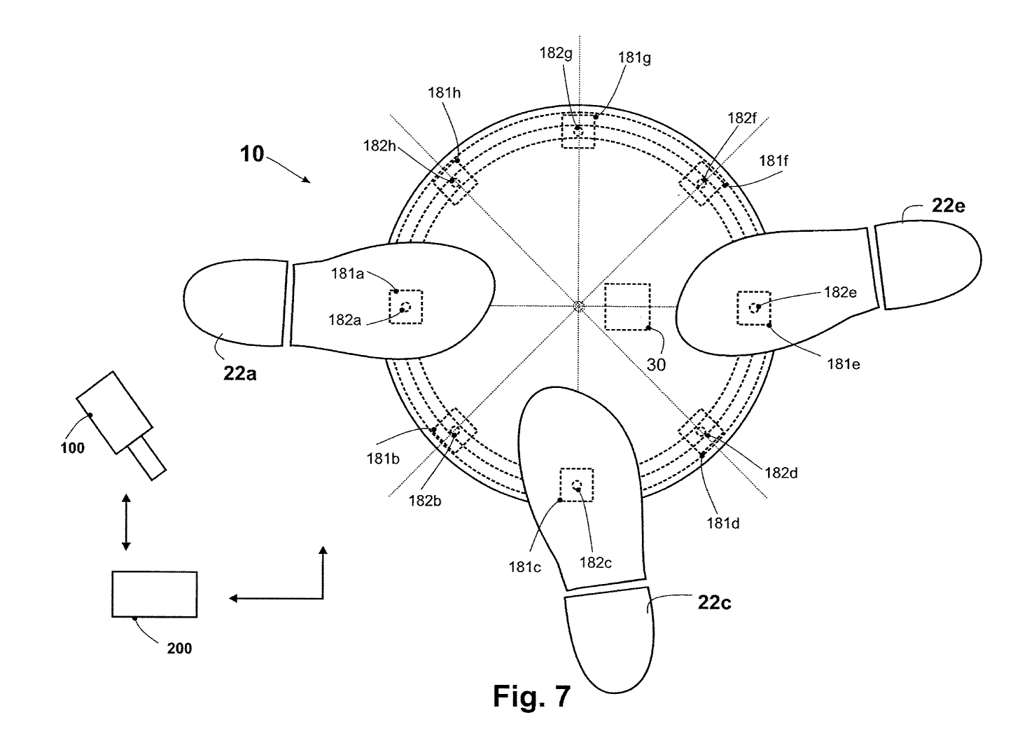

FIG. 7 shows the top view of the control device of FIG. 1, with three different foot positions. In particular, FIG. 7 shows a schematic representation of the control device, with three different foot positions 22a, 22c, 22e of a user or operator which may occur during a period of time of using the control device. Related to the actuation element 141 and the actuation sensors 181a-181h, the foot positioning is used during the calibration mode in order to define or determine a reference position (for example via the load point as described above) as the position of the user to assign the control functions or functions of the instrument to the corresponding areas of the actuation element and actuation sensors or vice versa.

It should be noted that “foot position” and “foot” can be used synonymously. Activation may also be performed by hand or by finger or by other kinds of touch or interactions.

The actuation system is preferably in communicative relationship with the actuation sensors. In the embodiment shown in FIGS. 1 and 2 or also 7, the actuation element 141 is preferably mechanically attached or attachable to the eight actuation sensors. In order to define the reference position in relation to the actuation element 141, the signals of or from the actuation sensors are used. Each sensor element produces either a zero signal, or a low or a high sensor signal depending on the local actuation load, that is, for example in response to an external force applied to the actuation element and thus to the corresponding actuation sensor(s). In one embodiment, the sensor elements are responsive to movement of the actuation element, thus, due to the external force, the actuation sensors can be actuated. In the case that a foot 22a of a user is pressing directly on or very close to a specific actuation sensor, e.g. 181a, a corresponding sensor signal S181a provided by said actuation sensor will be the highest signal. One or more actuation sensors 181b and/or 181h in the neighborhood of the specific actuation sensor 181a may give a little or lower signal as well, see FIG. 11. The multilevel strength signals can be replaced by digital 0/1 switch signals as well.

In the case that a user causes a single or pure sensor signal of one of the actuation sensor, for example of actuation sensor 181a, a load point or reference position is assigned to actuation sensor 181a. In the case of foot position 22c, the result is a S181c signal and the load point is assigned to the actuation sensor 181c. A similar assignment can be performed in respect of foot or foot position 22e. After assignment of the load point and functions, the calibration mode is ended or terminated and the actuation mode is started.

Defining or assigning the load point or reference position to one or more corresponding actuation sensors corresponds to the definition of a relative position between the control device and a user. In the calibration mode, a relative position between the control device and a user is defined, and, depending on the relative position between the control device and a user, the at least two actuation areas are preferably defined in relation to the at least two different functions, wherein the at least two different functions are assigned to the at least two different actuation areas of the actuation system, or wherein the at least two different functions are assigned to the at least two different ways of actuating an actuation element.

In the case that two actuation sensors, for example sensors 181a and 181b, are actuated in the calibration mode, the reference position L1 can also be assigned between the two actuation sensors 181a and 181b. The reference position defines for example a divide between the right and the left half of the actuation element 141.

In the case that the foot is not exactly positioned above one of the actuation sensors but between for example two actuation sensors, a mixed signal is generated and the specific signal mix of the actuation sensors is used to determine the foot positioning and thus the reference position.

FIG. 8 shows a cross-sectional view of a further embodiment of a control device embodying the present invention. Not all of the components inside the control device are illustrated in cross-sectional view. The Figure shows as an example the foot 22a, pressing a part of the actuation element 141. Thus, for example the actuation sensor or switch 181a is actuated.

The control device 10 comprises a mode switch system 24 preferably arranged in the center of the control device 10. The mode switch system 24 in this embodiment comprises a mounting element 24a, a mode switch element 24b arranged in an actuatable manner at the mounting element 24a, and a mode sensor element 24c for detection of an actuation of the mode switch element 24b. In this embodiment, the calibration mode is for example entered or selected by actuating the mode switch system 24, that is, the mode switch element 24b, then the calibration is performed and the load point/reference position L1 can be defined, for example based on an inclination of the actuation element 141 as described with other embodiments above. The mode sensor element 24c can be for example a pressure sensor, a level sensor, a weighing sensor and the like. Then, the calibration mode is ended or terminated and then the actuation mode may be entered, for example via mode switch system 24. In the actuation mode, further load points are determined in relation to the reference position or load point L1 and thus, corresponding function(s) of the instrument or device is/are performed.

FIG. 9 shows a top view of a further embodiment of a control device embodying the present invention. In particular, FIG. 9 shows an actuation system 14 comprising more than two, e.g. four actuation areas A11, A12, A13, A14, which are in this case realized by pressure contact elements, and thus, four sections which can be actuated for example by foot or hand pressure. There are inert areas or separator areas between the actuation areas or pressure contact elements which cannot be actuated. This helps to separate the actuatable actuation areas A11-A14. Thus, sensors 181b, d, f, h are not actuatable in this specific embodiment. The actuation areas A11-A14 cooperate with the actuation sensors 181a, c (not shown), e and g. The pressure contact elements are formed as predefined actuation areas and thus, are arranged on fixed positions. However, the assignment between function and corresponding area can be modified after starting a calibration mode.

Preferably, in the calibration mode the actuation area which is actuated first, for example for a predetermined period of time, causes a sensor signal, and thus, the load point or reference position is determined and/or defined.

In a further embodiment, a change of the mode, that is changing from calibration to actuation or from actuation to calibration, can be actively performed by a user, e.g. by voice control, by applying a specific predefined load pattern, e.g. pressing for example the actuation element for a specific period of time, for example longer than 5 to 7 seconds, or by operating or activating a mode switch system, such as system 24 shown in FIG. 8, that is for example by pressing the mode switch element 24b. In addition or as an alternative, the control device can be configured to automatically switch between the calibration mode and the actuation mode. The controller 200 can preferably comprise a timer or timer element 201, shown for example in FIG. 10; the timer element can also be arranged as a single element, preferably being in communication with the controller. The timer element is preferably configured to support the switch mechanism, that is switching between actuation mode or calibration mode. Thus, the timer element is preferably configured to process, preferably together with the controller, the predetermined periods of time, for example the first and/or second predetermined period of time, for selecting the calibration or actuation mode. The timer element may also be used for other settings.

FIG. 10 shows a top view of a further embodiment of a control device. In particular, FIG. 10 shows an embodiment with a double sensor equipment for each actuation area A11-A14. In other words, each actuation area, for example predefined pressure contact elements, is in communicative relationship with (or for example mounted on) more than one actuation sensor. As can be taken from FIG. 10, each actuation area cooperates with two separate sensor elements 181a, b; 181c, d; 181e, f; and 181g, h and thus, the symmetry of the applied contact force can be measured in order to detect the position of the foot with much higher precision and resolution, respectively. The actuation by foot (position) 22c results in a certain sensor signal distribution and thus the signal distribution can be reversely calculated and results in a detection of foot and foot position 22c, respectively.

FIG. 11 shows a diagram depicting the signals S181a, S181b, S181c, S181d, S181e, S181f, S181g, S181h, for the e.g. eight actuation sensors 181a-181h, of the control device 10 for example according to FIGS. 1 and 2, when an actuation is performed according to e.g. the foot position 22a, see FIG. 7. In this embodiment, actuation sensor 181a gives a high signal while the sensors 181b and 181h in the neighborhood give little side effect signals and the other sensors give zero signal. The sensor signals may be analog or digital 1/0 signals as well. The signal distribution is used to define the reference position respectively the relative position between the control device and the user. In the actuation mode, the same kind of actuation according to foot position 22a generates one or more sensor signals. The controller 200 then for example generates on the basis of the one or more sensor signals one or more corresponding function or functional signals, in order to control at least one function of an instrument or any other kind of device in communication with the control device.

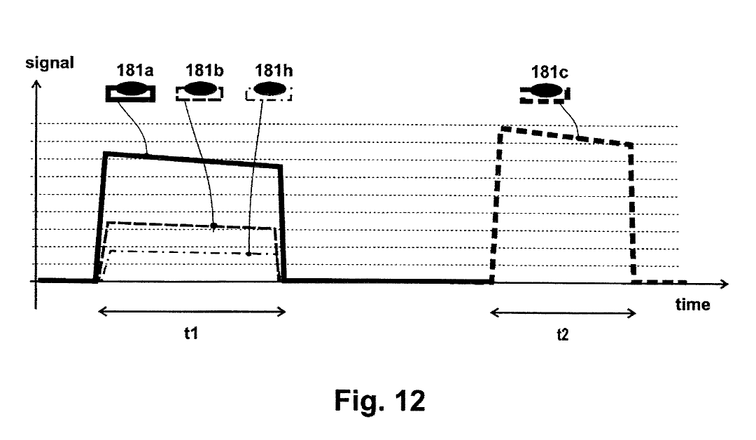

FIG. 12 shows a diagram depicting the signals of or from the actuation sensors 181a, b, h and c plotted over time, see for example FIG. 1. After entering the calibration mode by actuating the actuation element 141, the calibration mode in this embodiment is active for a first period of time t1. This is the calibration period t1 when the reference position is determined and/or defined and the one or more functions are assigned to the corresponding actuation areas. In the case that more than one sensors are actuated, the signal intensities and/or the signal sequences are used to determine the load point. In FIG. 12 the strongest signal of actuation sensor 181a determines and/or defines the reference position and the low (or lower) signal actuation sensors 181b and 181h are detected as side effects. Then a second period of time t2 is following. The second period of time t2 is an actuation period when a selected actuation area is for example pressed, and thus, for example at least sensor 181c is actuated, and the assigned control function is generated by controller 200 in order to control the connected instrument(s).

In the following, a preferred signal sequence is described in more detail. That is, at least parts of a preferred method are explained, how to operate a control device as described herein.

As can be taken for example from FIGS. 1 and 2, the control device preferably comprises the controller 200, externally or internally arranged in respect of the control device 10, so that the instrument or device 100 preferably communicates with the control device via the controller 200.

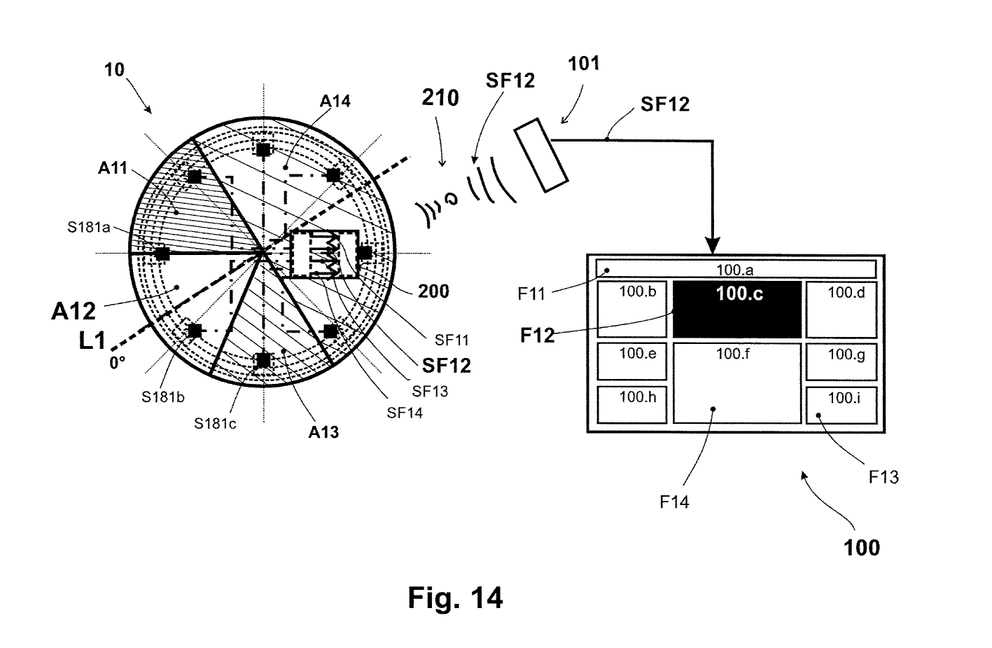

As can be taken from FIGS. 13 and 14 the signal transfer between the controller 200 and the device or devices 100 either transfers the sensor signals S181a . . . S181h or the function signals, for example SF11, SF12, SF13, SF14.

FIG. 13 is a representation showing an exemplary signal sequence for performing functions of an instrument in communication with a control device of the present invention. In particular, FIG. 13 shows the actuation mode with a control device comprising four actuation areas A11, A12, A13, A14.

FIG. 14 is a representation showing an exemplary signal sequence for performing functions of an instrument or device in communication with a control device of the present invention.

As shown for example in FIG. 13 one or more of the actuation sensors of the plurality of actuation sensors detect a specific kind of actuation, since the sensors are preferably responsive to the movement of the actuation element, and trigger(s) the actuation or calibration mode, as for example described above.

In the calibration mode, one or more of the plurality of actuation sensors 18, e.g. 181a . . . 181h, detect the kind of activation or actuation, preferably the kind of movement of the actuation system 14 and allow for calibrating the control device as for example described above, preferably by determining or measuring the inclination angle γ and, based on the inclination angle and the corresponding sensor signal(s), defining the load point or reference position, and defining the actuation areas, for example A11 and A12, see FIGS. 1 and 2. Then, an assignment between functions F11, F12 and actuation areas A11, A12 is performed. These steps are preferably controlled by the controller 200. As already indicated above, further actuation areas can be defined, for example A13, A14, . . . and further assignments between actuation areas and functions F13, F14, . . . can be performed (see for example FIG. 13).

In a preferred embodiment the areas A11, A12, . . . are assigned to function signals SF11, SF12, . . . and these function signals are assigned to functions F11, F12, . . . .

The signal sequence in the actuation mode is for example described in FIG. 13. During the actuation mode the plurality of sensor signals S181a S181h is created, wherein the actuation of area A12 in the embodiment according to FIG. 13 can primarily cause a specific sensor signal distribution S181a-h which is characteristic for the exact position and load direction of the actuation force that is applied to A12. As soon as the load point and/or load direction caused by actuation are different, a different sensor signal distribution will be created and detected. Nevertheless the translation from the sensor signal distribution to the functional signal SF12 may be the same as long as area A12 is actuated.

As shown in FIG. 13 and FIG. 14 the predefined actuation or assignment pattern (see FIG. 4c) with the actuation areas A11, A12, A13, A14 is already assigned to the actuation element 141 according to the reference point L1, which was defined during the calibration mode. According to the actuation of the area A12, e.g. by application of foot pressure, finally the function F12 is activated and/or controlled.

In one preferred embodiment the actuated element 141 and/or the actuation areas A11, A12, . . . create sensor signals S181 which are transferred directly to the at least one instruments 100.

In a further embodiment (see for example FIG. 14) the sensor signals S181a . . . S181h are translated to function signals SF 11 . . . 14 by means of the controller 200 and then the function or functional signal, e.g. SF12 is being transferred to the instrument 100 in order to trigger or control for example function F12. A signal receiver or receiving unit 101 for receiving the signals from the control device is preferably connected to the device 100, preferably via cable or wirelessly. The device can comprise the signal receiver 101.

In the case of activation or actuation of other actuation areas A11, A13, A14 of the device 10 the corresponding functions F11, F13, F14 are controlled.

The selection of a predefined assignment pattern from a group of predefined assignment patterns (see FIGS. 4a-d) allows the control of 2, 3, 4 or more functions of instrument(s) 100.

In a preferred embodiment the functions of the instrument(s) 100 are different functions of several instruments like F11 of a drill, F12 of a saw, F13 of a UV light equipment, etc. (see FIG. 13). In another preferred embodiment the functions of the instrument 100 are functionalities and control functions of different fields, such as fields 100.a, 100.b, 100.c, 100.d, 100.e, 100.f, 100.g, 100.h, 100.i of a user interface, shown e.g. on a video monitor device, e.g. on a dental control system (see FIG. 14).

In the actuation mode, the plurality of actuation sensors 18, e.g. 181a . . . 181h, detect the kind of activation or actuation of the actuation system 14, preferably via a corresponding actuation area, and allow for controlling an instrument 100 or device in communication with the control device 10 to perform at least one of the assigned functions F11, F12, . . . .

The actuation of a corresponding actuation area, e.g. A12, generates corresponding sensor signals S181a . . . S181h with different levels, see FIG. 11 and FIG. 13. These signals are preferably internally translated to a distinct function signal, e.g. SF12, preferably with the help of the controller 200. Each actuation results in preferably one distinct function signal, e.g. SF12. Then the function signal SF is transmitted preferably via wireless communication 210 to the instrument or device 100 and thus, the function F12 is performed. It is also possible to generate signals SF11, SF13 and/or SF14 and to perform functions F11, F13 and/or F14.

In other words, a specific signal pattern S181a . . . S181h can be interpreted by the controller 200 as an explicit and distinct actuation of one of the actuation areas, e.g. A12, and this causes the generation of a functional signal, e.g. SF12, which is transferred to the instrument or device, so that the assigned function F12 is performed.

That is, the controller 200 is preferably configured to process one or more sensor signals, e.g. S181a-S181h, to create at least one functional signal SF and/or to transmit the functional signal(s) to the instrument 100 or any other kind of device.

The transmission of the signal(s) can be performed wirelessly, for example via radio frequencies, or can be performed via corresponding cables or wires.

In further embodiments, where the control device 10 operates alternatively in the calibration or gauge mode on the one hand and in the control or actuation mode on the other hand the control device 10 is preferably configured to remain in the control or actuation mode as long as the control device 10 does not change its position and, thus, its angular position relative to the user. To this end, the control device 10 in these preferred embodiments furthermore preferably comprises means for detecting any change of position of the control device 10. Any substantial change of position of the control device 10 detected by these means or device will result in a transition to the calibration or gauge mode. Preferred means for detecting any change of position of the control device 10 comprise at least one ball rotatably supported within the base element 12 substantially functioning like a computer mouse ball. However, the person skilled in the art is well aware of various other means that could be used for detecting any change of position of the control device 10, such as infrared laser diodes as used in an optical computer mouse or any other motion sensor.

In further embodiments, where the control device 10 operates alternatively in the calibration or gauge mode on the one hand and in the control or actuation mode on the other hand, the control device 10 furthermore can comprise means for visually indicating to a user whether the control device 10 currently operates in the calibration or gauge mode or in the control or actuation mode, such as an indication system. Preferably, a plurality of light sources, for example light emitting diodes (LEDs), are provided on the actuation surface 142 of the actuation element 141 at positions that correspond to and are aligned with the positions of the actuation sensors 181a-181h on the base element 12 such that each LED corresponds to an actuation sensor. These LEDs not only allow the user to determine the position of an actuation sensor 181a-181h located within the cavity 20 defined between the base element 12 and the actuation element 141, see for example FIGS. 1 and 2, but also indicate whether the control device 10 currently operates in the calibration or gauge mode or in the control or actuation mode. For instance, in the calibration or gauge mode the control device 10 could be configured such that all LEDs blink concurrently at certain intervals indicating to the user that the control device 10 has not been calibrated yet and is ready for calibration. After a calibration of the control device 10 according to a preferred calibration mechanism described further above, wherein by exerting a force at the exemplary position of the actuation surface 142 of the actuation element 141 marked L1 in FIG. 1 the actuation sensors lying on the “left side” of the notional line L running in the plan view of FIG. 1 from the point L1 through the center of the control device 10, i.e., the actuation sensors 181a and 181f-181h, have been assigned to a first function of an instrument in communication with the control device 10 and the actuation sensors lying on the “right side” of this notional line L, i.e., the actuation sensors 181b-181e, have been assigned to a second function thereof, the control device 10 could be configured such that the LEDs corresponding to the actuation sensors lying on the “left side” of the notional line L, i.e., the actuation sensors 181a and 181f-181h, emit light according to a different temporal pattern than the LEDs corresponding to the actuation sensors lying on the “right side” of this notional line L, i.e., the actuation sensors 181b-181e. For instance, the LEDs corresponding to the actuation sensors 181a and 181f-181h and the LEDs corresponding to the actuation sensors 181b-181e could emit light pulses alternately or one of these groups of LEDs could emit light continuously, whereas the other group of LEDs does not emit any light. Such configurations of the control device 10 in the control or actuation mode help the user to discern which parts of the actuation surface 142 of the actuation element 141 he or she has to actuate in order to trigger the various functions of an instrument in communication with and controlled by the control device 10.

In the case of a wireless device 10 without any wire or cable, the orientation of the control device is usually not visible or recognizable. Therefore, in a further embodiment, the reference position, adjusted in the calibration mode, can be visually indicated, for example by means of light sources as discussed above.

The indication device or system is preferably configured as an optical indication system and is configured to indicate the home position and/or a load point/reference position of the control device. Also subsequent load points determined in the actuation mode can be indicated. In an embodiment, a ring or circular arrangement of a plurality of LEDs can be provided, wherein the arrangement is configured such that the LED which corresponds to the reference position or which is assigned to the reference position emits light. The indication device may also be a display device displaying or indicating the kind and the position of the selected predefined assignment pattern.

In an embodiment, the indication system, for example the optical indication system, is configured to indicate the kind of assignment pattern A11 . . . A14 and/or the position of the assignment pattern and/or the position of the reference point L1. The indication system may also indicate where the actuation areas A11 . . . A14 have the highest sensitivity ore accuracy in order to help the user to perform the activation of the control device properly.

The control device 10 of this invention may be used to control the operation of various instruments and machines, such as electrocardiogram machines, X-ray machines, surgical cutting instruments, endoscopic and laparoscopic tools, blood analyzers, diagnostic tools, dental chairs, dental irrigators, dental air polishing and prophylaxis systems, dental drills, endodontic and periodontic handpieces, and other dental equipment. Other instruments, machines or devices may be used with the control device as well, for example music instruments etc.

Preferably, the control device 10 is used to operate a dental/medical or any other suitable instrument or device or monitors in a wireless, remote control system. In such a system, the control device 10 may include a transmitter or transceiver that transmits for example a radio frequency (RF) signal to a RF receiver in an optional base unit of the dental/medical instrument, which receives the signal. Wireless information including, for example, identification codes, equipment status, alarm messages, and the like may be sent back and forth between the control device 10 and the dental/medical instrument using such an RF transceiver. It is recognized that wireless communication systems, other than RF systems, could be used. For example, infrared or ultrasound communication systems could be used.

Alternatively, the control device 10 according to the present invention may be used to operate a dental/medical instrument or any other instrument in a hard-wired system. In such a system, the control device 10 is connected to an optional base unit of the dental/medical instrument by a connector cable extending from the control device 10. Control signals or function signals are sent from the control device 10 or its optional control unit or controller to the dental/medical instrument or its optional base unit via the connector cable.

The present invention as described in detail above is not limited to the particular devices, uses and methodology described as these may vary. For instance, although the present invention has been described above in the context of preferred embodiments of a foot switch, it can also be applied advantageously to switches operated by other means, such as the hands of a user. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### LIST OF REFERENCE SIGNS

10 control device
12 base element
14 actuation system
141 actuation element, (convex) contact plate, dome
142 actuation surface
143 peripheral shoulder
144 annular skirt
16a-d support element(s)
161a-d telescoping or support post(s)
162a-d spring elements
18 sensor system
181a-h actuation sensors, switch posts
182a-h pin(s) of actuation sensor(s)
20 cavity
22a, c, e foot, foot positioning of a user, operator
24 mode switch system
24a mounting element
24b mode switch element
24c mode sensor element
30 power supply, power source
100 instrument
100.a . . . 100.i fields of user interface
101 signal receiver
200 controller, computer unit, processor of the control device
201 timer, timer element
210 wireless signal transmission/communication
A11, A12, A13, A14 actuation areas
F force, applied by a user
F11-14 functions controlled by the control device
L notional line, load line
L0 home position of the control device
L1 load point/reference position
0° home position of a predefined assignment pattern
PI inclination plane
PB base plane
S central axis, e.g. symmetry axis
S′ inclined axis
S181a-h sensor signal, actuation signal
SF11-14 functional or function signals, created by controller 200
t1 first period of time, calibration period
t2 second period of time, actuation period
γ inclination angle
α calibration angle

1. A method of operating a control device for controlling at least two different functions of an instrument in communication with the control device, comprising the following steps:
calibrating the control device in a calibration mode by assigning the at least two different functions to at least two different ways of actuating an actuation element; and
controlling the instrument in communication with the control device in an actuation mode to perform at least one of the at least two different functions defined in the calibration mode by an actuation of the actuation element.

2. The method of claim 1, wherein the at least two different functions are assigned to the at least two different ways of actuating the actuation element by an actuation of the actuation element.

3. The method of claim 1, wherein the at least two different functions are assigned to the at least two different ways of actuating the actuation element by positioning a user's foot relative to the control device.

4. The method of claim 1, further comprising detecting an actuation of the actuation element, wherein the actuation results in a motion of the actuation element towards a base element of the control device floatingly supporting the actuation element.

5. The method of claim 1, further comprising exerting a force onto the actuation element for defining a reference position of the control device as a basis for assigning the at least two different functions to the at least two different ways of actuating the actuation element.

6. The method of claim 1, further comprising exerting a force onto the actuation element at two different locations or at at least two different locations of an actuation surface of the actuation element for actuating the actuation element in the actuation mode.

7. A method of operating a control device for controlling at least two different functions of at least one instrument in communication with the control device, the control device being operable in a calibration mode and in an actuation mode, the method comprising the following steps:
(a) providing an actuation system for actuating the control device,
(b) providing a sensor system for detecting an actuation of the actuation system,
(c) selecting the calibration mode for defining a relative position between the control device and a user, and,
depending on the relative position between the control device and a user, assigning the at least two different functions to at least two different actuation areas of the actuation system, and
(d) selecting the actuation mode for controlling the at least two different functions by the at least two actuation areas.

8. The method of claim 7, further comprising:
depending on the relative position between the control device and the user, defining the at least two actuation areas of the actuation system.

9. The method of claim 7, further comprising assigning the at least two different functions to the at least two different actuation areas, provided that not more than one function is assigned to one actuation area.

10. The method of claim 7, further comprising:
actuating the actuation system to select at least one of the following: the calibration mode and the actuation mode.

11. The method of claim 7, wherein the actuation of the actuation system results in a motion of the actuation system towards a base element of the control device floatingly supporting the actuation system.

12. The method of claim 7, further comprising defining the relative position between the control device and the user by an actuation of the actuation system.

13. The method of claim 7, further comprising defining the relative position between the control device and the user by positioning a user's foot relative to the control device.

14. The method of claim 7, further comprising defining the relative position between the control device and the user by exerting a force onto the actuation system for defining a reference position of the control device as a basis for assigning the at least two different functions to the at least two different actuation areas.

15. The method of claim 7, further comprising selecting a predefined assignment pattern from a plurality of predefined assignment patterns in order to define the at least two actuation areas.

16. The method of claim 7, further comprising exerting a force onto the actuation system at the at least two different actuation areas for actuating the actuation system for controlling the at least two different functions.

17. A control device for controlling at least two different functions of an instrument in communication with the control device, the control device comprising an actuation element configured to be actuated in at least two different ways, wherein the control device is configured to be operated in a calibration mode and an actuation mode such that in the calibration mode a respective function of the at least two functions can be assigned to the at least two different ways of actuating the actuation element and such that in the actuation mode an actuation of the actuation element of the calibrated control device in either one of the at least two different ways of actuating the actuation element actuates the function assigned to the respective way of actuating the actuation element.

18. A control device for controlling at least two different functions of at least one instrument in communication with the control device, the control device being operable in a calibration mode and in an actuation mode, the control device comprising:
a base element,
an actuation element supported by the base element, and
a plurality of actuation sensors distributed on the base element about a central axis,
the plurality of actuation sensors being responsive to movement of the actuation element.

19. The control device of claim 18,
wherein in the calibration mode,
a relative position between the control device and a user is defined,
depending on the relative position between the control device and a user, the at least two different functions are assigned to at least two different actuation areas of the actuation element, and
wherein in the actuation mode, the at least two actuation areas are actuatable to control the at least two different functions.

20. The control device of claim 19, further comprising:
depending on the relative position between the control device and the user, the at least two actuation areas which form the actuation element are definable.

21. The control device of claim 18, wherein the base element is a planar base element.

22. The control device of claim 18, wherein the plurality of actuation sensors are symmetrically distributed on the base element about the central axis.

23. The control device of claim 18, wherein the actuation element is floatingly supported by the base element.

24. The control device of claim 18, wherein the actuation element is actuatable to select at least one of the following: the calibration mode and the actuation mode.

25. The control device of claim 18, further comprising a controller configured to perform at least one of the following: to process one or more sensor signals of the plurality of actuation sensors, to generate one or more function signals and to transfer the one or more function signals to the instrument or to the at least one instrument or to a controller of the instrument.

26. The control device of claim 19, wherein the control device is configured to select a predefined assignment pattern from a plurality of predefined assignment patterns in order to define the at least two actuation areas of the actuation element.

27. The control device of claim 18, wherein a cavity is defined between the base element and the actuation element and the plurality of actuation sensors are arranged within the cavity.

28. The control device of claim 18, wherein the plurality of actuation sensors are configured to detect an actuation of the actuation element, wherein the actuation of the actuation element results in a motion of the actuation element towards the base element.

29. The control device of claim 18, wherein the actuation element is supported above the plurality of actuation sensors and actuatable by being movable toward the plurality of actuation sensors in response to an external force applied to the actuation element.

30. The control device of claim 18, wherein the actuation element is supported above the plurality of actuation sensors by at least one support element or a plurality of support elements.
